(19) United States
(12) Patent Application Publication
(10) Pub. No.: US 2021/0355434 A1
(43) Pub. Date: Nov. 18, 2021

(54) Methods of Producing Cannabinoids

(71) Applicant: Basil Mohammad Hantash, San Juan, PR (US)

(72) Inventor: Basil Mohammad Hantash, San Juan, PR (US)

(73) Assignee: EVN Holdings LLC, Sheridan, WY (US)

(21) Appl. No.: 17/318,651

(22) Filed: May 12, 2021

Related U.S. Application Data
(60) Provisional application No. 63/024,770, filed on May 14, 2020.

(51) Int. Cl. C12N 1/20; C12P 7/22

(52) U.S. Cl. CPC C12N 1/205; C12P 7/22

(57) ABSTRACT

Modified bacteria and methods for the construction of metabolic pathways inside bacteria to produce biomolecules including cannabinoids, cannabinoid precursors, and cannabinoid derivatives are disclosed.

### BACKGROUND

Cannabis has been in use by humans for millennia, due to the multiplicity of its benefits to humans, including the considerable value and utility of its fiber, the nutritional value of its seeds, and the medicinal value of its floral parts and products made from them. Currently the genus is under intense legal commercialization in the United States as industrial hemp for a variety of purposes including biodegradable plastics and building materials, clothing, paper, food, fuel and medicines.

Cannabidiol (CBD) extracted from Cannabis is widely used in over-the-counter medicines and topical treatments, and is also the active ingredient in the FDA-approved drug Epidiolex. CBD is just one of at least dozens—perhaps hundreds—of cannabinoids endogenous to Cannabis, tetrahydrocannabinol (THC) being the other cannabinoid that is most well-known. The cannabinoids as a group interact with the human endocannabinoid receptors, which are distributed in the brain and throughout the body. The study of the endocannabinoid system (ECS) in humans and other mammals is an area of increasing interest and holds tremendous promise for the future of medicine. See, e.g., Russo (2019). Cannabis and Pain, Pain Medicine, 20(10): 1093/pm/pnz227; and Russo (2016). Clinical Endocannabinoid Deficiency Reconsidered: Current Research Supports the Theory in Migraine, Fibromyalgia, Irritable Bowel, and Other Treatment-Resistant Syndromes, Cannabis Cannabinoid Res. 1(1): 154-165.

Cannabinoids and cannabinoid precursors can be effective for the treatment of a wide range of medical conditions, including neuropathic pain, AIDS wasting, anxiety, epilepsy, glaucoma, and cancer, amongst others. Current methods of producing cannabinoids include the growth of the cannabis plant and industrial production of synthetic cannabinoids.

### BRIEF DESCRIPTION OF THE DRAWINGS

FIG. 1 is a flowchart of one embodiment of the process.

### DETAILED DESCRIPTION

The invention relates to the use of the organism Vibrio natriegens as a host for biotechnological applications, particularly as a host for the construction, maintenance, manipulation, and/or propagation of recombinant DNA constructs (including synthetic or semi-synthetic DNA constructs); for protein expression; for metabolic engineering; for the preparation of cellular extracts for cell-free biology (e.g., cell-free protein synthesis, in vitro enzymatic catalysis, DNA replication, and RNA transcription); and as a chassis for synthetic biology applications. Applications related to molecular biology, synthetic biology, and metabolic engineering can be accelerated using the V. natriegens host due to its rapid growth rate and nutritional versatility.

Additional information can be found in Wang, Zheng et al. “Melanin Produced by the Fast-Growing Marine Bacterium Vibrio natriegens through Heterologous Biosynthesis: Characterization and Application.” Applied and environmental microbiology vol. 86, 5 e02749-19. 18 Feb. 2020, doi:10.1128/AEM.02749-19; Lee, Henry H et al. “Functional genomics of the rapidly replicating bacterium Vibrio natriegens by CRISPRi.” Nature microbiology vol. 4, 7 (2019): 1105-1113. doi:10.1038/s41564-019-0423-8; Pfeifer, Eugen et al. “Generation of a Prophage-Free Variant of the Fast-Growing Bacterium Vibrio natriegens.” Applied and environmental microbiology vol. 85, 17 e00853-19. 14 Aug. 2019, doi:10.1128/AEM.00853-19; Hoffart, Eugenia et al. “High Substrate Uptake Rates Empower Vibrio natriegens as Production Host for Industrial Biotechnology.” Applied and environmental microbiology vol. 83, 22 e01614-17. 31 Oct. 2017, doi:10.1128/AEM.01614-17; Weinstock, Matthew T et al. “Vibrio natriegens as a fast-growing host for molecular biology.” Nature methods vol. 13, 10 (2016): 849-51. doi:10.1038/nmeth.3970; and Maida, Isabel et al. “Draft Genome Sequence of the Fast-Growing Bacterium Vibrio natriegens Strain DSMZ 759.” Genome announcements vol. 1, 4 e00648-13. 22 Aug. 2013, doi:10.1128/genomeA.00648-13. All the foregoing are hereby incorporated by reference in their entirety.

Vibrio is a genus of Gram-negative, facultative anaerobic bacteria possessing a curved-rod shape. In some embodiments, Vibrio sp. comprises one or more of the following Vibrio species: adaptatus, aerogenes, aestivus, aestuarianus, agarivorans, albensis, alfacsensis, alginolyticus, anguillarum, areninigrae, artabrorum, atlanticus, atypicus, azureus, brasiliensis, bubulus, calviensis, campbellii, casei, chagasii, cholera cincinnatiensis, coralliilyticus, crassostreae, cyclitrophicus, diabolicus, diazotrophicus, ezurae, fischeri, fluvialis, fortis, furnissii, gallicus, gazogenes, gigantis, halioticoli, harveyi, hepatarius, hippocampi, hispanicus, hollisae, ichthyoenteri, indicus, kanaloae, lentus, litoralis, logei, mediterranei, metschnikovii, mimicus, mytili, natriegens, navarrensis, neonates, neptunius, nereis, nigripulchritudo, ordalii, orientalis, pacinii, parahaemolyticus, pectenicida, penaelcida, pomeroyi, ponticus, proteolyticus, rotiferianus, ruber, rumoiensis, salmonicida, scophthalmi, splendidus, superstes, tapetis, tasmaniensis, tubiashii, vulnificus, wodanis, and xuii, as well as any other species of the genus Vibrio.

Vibrio natriegens is a Gram-negative marine bacterium. It was first isolated from salt marsh mud and is a halophile requiring about 2% NaCl for growth. It reacts well to the presence of sodium ions which appear to stimulate growth in Vibrio species, to stabilize the cell membrane, and to affect sodium-dependent transport and mobility. Under optimum conditions, and all nutrients provided, the doubling time of V. natriegens can be less than 10 minutes. Its rapid growth rate (the fastest known doubling time of any organism), its ability to thrive in inexpensive, defined media, its ability to serve as a drop-in replacement for E. coli strains for common lab processes, its unique genome architecture (which can be leveraged to facilitate the cloning of large DNAs), and the potential to leverage natural transformation and conjugation as genetic engineering tools makes V. natriegens an attractive host. It has the potential to dramatically speed up standard workflows, as well as to make possible projects that are otherwise not feasible when working with the current state-of-the-art alternatives.

Vibrio sp. has several advantages over other bacteria for many molecular biology applications. One such advantage is the rapid growth/doubling rate of Vibrio sp. One of the most time-intensive steps in modern biotech workflows is waiting for the host to grow to a sufficient density before DNA/protein/product can be recovered or the phenotype can be assessed. As dramatic time savings have been realized in other areas of biotech workflows (e.g., sequencing, bioinformatic analysis, high-throughput assays, etc.), growth of the host has become a significant bottleneck. E. coli is considered to have one of the quickest growth rates relative to other organisms used in the biotech sector, and this has been one of its strengths. Because V. natriegens has a growth rate 2-3× faster than commonly used E. coli strains, it is able to effect a dramatic reduction in the time necessary for the host to grow, and will accelerate research efforts. In some embodiments of the invention, the growth rate (expressed as the time required for a culture density to double) of Vibrio sp. is about 10 minutes. In some embodiments, the growth rate of a genetically engineered Vibrio sp. is about 5 minutes to 30 minutes. In some embodiments the Vibrio sp. organisms of the invention have a doubling time of less than 15 minutes or less than 14 minutes or less than 13 minutes or less than 12 minutes or less than 11 minutes or less than 10 minutes, or less than 9 minutes, or less than 8 minutes, or less than 7 minutes, or less than 6 minutes, or less than 5 minutes, or less than 4 minutes. The doubling time can be achieved by the organism in a rich medium, for example, a medium rich in nitrogen and carbon. In some embodiments, the doubling times described can be achievable in any of the media described herein. For example, the doubling times disclosed can be achieved in an LB broth, in LB agar, in Nutrient Broth+1.5% NaCl, in Brain Heart Infusion (with or without salts), Brain Heart Infusion Agar (with or without salts), SSG agar, 2×YT+salts+glucose+phosphate buffer, Vegitone Infusion Broth (with optional salts), LB+salts+glucose+phosphate buffer. Doubling times can be measured at the flat or log portion of the curve, so long as the same portion of the curve is used consistently

Another advantage of Vibrio sp. is the amount of exogenous DNA constructs that can be harbored therein. Large scale genetic engineering/synthetic genome construction efforts require the assembly, manipulation, and maintenance of large pieces of recombinant DNA, tasks which are carried out in a genetically tractable host (such as E. coli) before delivery of the engineered DNA to the final host organism. Currently, most of this work is carried out in E. coli, but as projects become more ambitious, the limitations of this species are becoming apparent. It has been observed that with current technologies, E. coli is capable of harboring exogenous DNA constructs of no more than 500 kb (and in some cases much less depending on the properties of the DNA being cloned) on a bacterial artificial chromosome, which is a serious limitation for synthetic genome/large pathway construction efforts. This has necessitated the development of novel hosts as cloning platforms such as Saccharomyces cerevisiae and Bacillus subtilis. While these hosts have the advantage of being able to take up and stably propagate large (Mb-sized) fragments of exogenous DNA, they have their own disadvantages, with Saccharomyces cerevisiae growing much slower than E. coli (^{˜}3× slower), and both species being incompatible with standard laboratory techniques and being very difficult to recover DNA from. Vibrio sp can accept constructs over 500 kb. In some embodiments, the constructs are at least 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000 kb or more.

An additional advantage is the compatibility of Vibrio sp. with standard lab protocols: Unlike other niche organisms, which often require specialized techniques/methods, Vibrio sp. is compatible with many standard cloning vectors, growth media, workflows and commercially-available kits developed for E. coli or recovering DNA. This compatibility with standard tools/reagents/methods lowers the barrier to adoption by labs that are currently dependent on E. coli, allowing for drop-in replacement.

A further advantage is the nutritional versatility of Vibrio sp. Vibrio sp. has extreme nutritional versatility, allowing it to grow on a range of different growth media, including inexpensive, minimal media. Coupled with its rapid growth rate, this feature can allow for industrial scale production of biomolecules (e.g., therapeutic proteins, commodity chemicals, etc.) cheaper and faster than the state of the art. In the instant invention, V. natriegens and a genetically engineered V. natriegens are capable of growing under a variety of nutrient and temperature conditions. V. natriegens is fairly promiscuous in terms of the carbon sources that it can utilize (much more so that E. coli). Embodiments of the invention can relate to a fermentation process using V. natriegens that leverages several different low-cost carbon feedstocks. This versatility can be a major benefit if driving towards commodity chemicals (of which cannabinoids are). In general, productive medias can include high salt content (>10 g/L NaCl). A variety of carbon sources can be used depending on cost considerations.

Members of Vibrionaceae have a unique two-chromosome genome, that can be referred to as Chromosome 1 and Chromosome 2. A genetically engineered V. natriegens can be constructed with a single, large chromosome which incorporates the essential features from the smaller chromosome into the large chromosome. In this genetically engineered V. natriegens, the now “free” chromosomal machinery can be leveraged as a vector for cloning large DNAs/pathways. The smaller second chromosome of V. natriegens can be capable of replicating/maintaining about a 2 Mb fragment of DNA showing that the use of chromosome 2 as a cloning vector can allow for the rapid and robust propagation of large exogenous DNA molecules (e.g., synthetic or semi-synthetic chromosomes for ultimate use in other organisms, or novel pathways/genetic elements for use in V. natriegens itself) as well as the production of polypeptides and biomolecules.

The invention relates to methods to enable the construction of metabolic pathways inside V. natriegens to produce bespoke cannabinoids, cannabinoid precursors, cannabinoid derivatives, or cannabinoid precursor derivatives from simple precursors such as sugars and carboxylic acids. One or more heterologous nucleic acids disclosed herein encoding one or more polypeptides disclosed herein can be introduced into host microorganisms allowing for the stepwise conversion of inexpensive feedstocks, e.g., sugar, into final products: cannabinoids, cannabinoid precursors, cannabinoid derivatives, or cannabinoid precursor derivatives. These products can be specified by the choice and construction of expression constructs or vectors comprising one or more heterologous nucleic acids disclosed herein, allowing for the efficient bioproduction of chosen cannabinoid precursors; cannabinoids, such as THC or CBD and less common cannabinoid species found at low levels in Cannabis; or cannabinoid derivatives or cannabinoid precursor derivatives. Bioproduction also enables synthesis of cannabinoids, cannabinoid derivatives, cannabinoid precursors, or cannabinoid precursor derivatives with defined stereochemistries, which is challenging to do using chemical synthesis.

The nucleic acids can include those encoding a polypeptide having at least one activity of a polypeptide present in the cannabinoid biosynthetic pathway, such as a geranylpyrophosphate:olivetolate geranyltransferase (GOT) polypeptide (e.g., a CsPT4 polypeptide), responsible for the biosynthesis of the cannabinoid CBGA; a tetraketide synthase (TKS) polypeptide; an olivetolic acid cyclase (OAC) polypeptide; and a CBDA or THCA synthase polypeptide. Nucleic acids can include those encoding a polypeptide having at least one activity of a polypeptide involved in the synthesis of cannabinoid precursors. These polypeptides include, but are not limited to, polypeptides having at least one activity of a polypeptide present in the mevalonate pathway; polypeptides that generate acyl-CoA compounds or acyl-CoA compound derivatives (e.g., an acyl-activating enzyme polypeptide, a fatty acyl-CoA synthetase polypeptide, or a fatty acyl-CoA ligase polypeptide); polypeptides that generate GPP; polypeptides that generate malonyl-CoA; polypeptides that condense two molecules of acetyl-CoA to generate acetoacetyl-CoA, or pyruvate decarboxylase polypeptides.

The invention relates to generation of cannabinoid precursor derivatives or cannabinoid derivatives, as well as cannabinoids or precursors thereof, with polypeptides that generate acyl-CoA compounds or acyl-CoA compound derivatives. In some embodiments, genetically modified bacteria are modified with one or more heterologous nucleic acids encoding a polypeptide that generates acyl-CoA compounds or acyl-CoA compound derivatives. These polypeptides can permit production of hexanoyl-CoA, acyl-CoA compounds, derivatives of hexanoyl-CoA, or derivatives of acyl-CoA compounds. In some embodiments, hexanoic acid or carboxylic acids other than hexanoic acid are fed to genetically modified host cells expressing a polypeptide that generates acyl-CoA compounds or acyl-CoA compound derivatives (e.g., are present in the culture medium in which the cells are grown) to generate hexanoyl-CoA, acyl-CoA compounds, derivatives of hexanoyl-CoA, or derivatives of acyl-CoA compounds. These compounds are then converted to cannabinoid derivatives or cannabinoid precursor derivatives, as well as cannabinoids or precursors thereof, via one or more polypeptides having at least one activity of a polypeptide present in the cannabinoid biosynthetic pathway or involved in the synthesis of cannabinoid precursors.

In some embodiments, the present invention provides genetically engineered Vibrio sp. bacteria comprising one or two altered, rearranged, or minimized chromosomes. In some embodiments, the essential elements from Chromosome 2 are alternatively located on Chromosome 1. In some embodiments, the engineered bacteria contain a single chromosome comprising the essential features of Chromosome 1 and 2. In some embodiments, the engineered Vibrio sp. are generated by knocking out and/or knocking in appropriate genes in order to generate a desired engineered Vibrio sp. The genes can be related to enzymes of a cannabinoid or terpenoid biosynthesis pathway. The genes can be related to regulators of enzymes of a cannabinoid, cannabinoid derivative or terpenoid biosynthesis pathway. In some embodiments, the engineered Vibrio sp. produces at least four substances found in a Cannabis plant. The substances can be, for example, cannabinoids or terpenes, or derivatives of the same. The compound can be, for example, pentyl, propyl, C-4, C-1 and monomethylether constituents of cannabinoid families, including but not limited to acidic and neutral forms of the cannabigerol (CBG), cannabichromene, cannabidiol (CBD), delta-9-tetrahydrohydrocannabinol, delta-8-tetrahydrohydrocannabinol, cannabielsoin, cannabinol and cannabinodiol cannabinoid classes; and, cis and trans terpenoids or terpenes, including but not limited to myrcene, limonene, linalool, ocimene, beta-pinene, alphapinene, beta-caryophyllene, alpha-caryophyllene, delta-3-carene, ganmia-bisabolene, alpha-farnesene, beta-fenchol, guajol, alpha-guaiene, terpinolene, beta-eudesmol, alphabergamotene, epi-alpha-bisabolol and caryophyllene oxide; and/or the like. In some embodiments, the engineered Vibrio sp. produces 4, 5, 6, 7, 8, 9, 10, 11, 12, 13, 14, 15, 16, 17, 18, 19 or more substances found in a Cannabis plant. In some embodiments the knock in and/or knock out and/or sequence inversion is enabled through the enzyme activity of a recombinase, such as, for example, Cre recombinase. In some embodiments, the Cre recombinase activity utilizes known lox sites compatible with Cre recombinase. In some embodiments, the knock in and/or knock out is enabled through the enzyme activity of a nuclease, such as, for example, Type II CRISPR Cas9. In some embodiments, the knock in and/or knock out is enabled through the use of a homologous recombination vector containing regions of sequence homology to a region in the genome where an insertion or deletion is desired. In some embodiments, the homologous recombination vector is incorporated by a single cross-over event. In some embodiments, the homologous recombination vector is incorporated by a double-cross-over event. In some embodiments the knock in and/or knock out event is enabled through use of an integrase, such as, for example, PhiC31 or bxb 1. In some embodiments, the knock in and/or knock out is enabled through the use of a suicide vector. In some embodiments the vector is assembled in vitro and subsequently transformed and amplified in E. coli. In some embodiments the vector is assembled in S. cerevisiae. In some embodiments, the amplified vector is introduced into V. natriegens by conjugation, electroporation, chemical competence, biolistics, transduction, or via natural competence.

In some embodiments, the amplified vector is introduced by bacterial conjugation, natural competence, electroporation, chemical transformation, or the like. In some embodiments, the electroporation is nucleofection. Chemical transformation can include Calcium Chloride, Super Optimal broth with Catabolite repression (SOC), or the like.

For making changes to or introducing novel genes into the genome, natural transformation can be a suitable approach. For transforming plasmid DNA into the strain, electroporation or chemical transformation can be very well suited and electroporation can be especially well suited. It should be noted that transformation efficiency is highly strain dependent. In some embodiments, the genetically engineered Vibrio sp. comprises altered chromosomes or a combined single chromosome, either of which has been minimized, whereby non-essential genes and nucleic acid sequences have been removed. Non-limiting examples of non-essential genes include exonucleases, endonucleases, methylases, nucleases, restriction enzymes, complete restriction-modification systems, or any combination thereof. Non-essential genes or genetic elements can be identified bioinformatically or experimentally. Bioinformatic identification can involve comparing multiple wild-type V. natriegens strain genomes and identifying genes or nucleic acid sequences that are not consistently present in all strains. Experimental identification of non-essential genes can be achieved by transposon bombardment or other insertional mutagenesis screens that will produce multiple random integration mutants. By sequencing the genes disrupted in these viable mutants, non-essential genes will be identified. In some embodiments, the non-essential genes can be sequentially removed by homologous recombination-based techniques. In some embodiments, the minimization can be achieved sequentially through known techniques, such as multiplex automated genome engineering (MAGE) or hierarchical conjugative assembly (CAGE). Embodiments of the invention relate to removing genetic material that is not essential in order to reduce energy consumption by the ribosomal system so that energy is used for promoting rapid growth and highest yield of desired metabolic pathway.

Bacterial cells function such as genome replication and host restriction systems are regulated by epigenetic modifications, including but not limited to, DNA methylation and histone deacetylation. In some embodiments, epigenetic modifying agents are employed to enhance genome replication, overcome host restriction systems, enhance foreign DNA expression, and/or to enhance other bacterial cell functions that are involved in efficient production of the desired chemical, in particular, cannabinoids or a cannabinoid derivatives.

In some embodiments, the invention relates to genetically engineered Vibrio sp. bacteria comprising an altered chromosomal arrangement. In some embodiments, one or more non-essential elements are removed from Chromosome 1 and/or Chromosome 2. In some embodiments, one or more elements from Chromosome 2 are alternatively located on Chromosome I. In some embodiments, the genetically engineered Vibrio sp. comprises a single chromosome. In some embodiments, the single chromosome contains key, useful, or essential genomic elements from Vibrio sp. Chromosome 1 and 2. In some embodiments, non-limiting examples of an essential element is a gene required for a function selected from the group consisting of metabolism, DNA replication, transcription, translation, cellular structural maintenance, transport processes into or out of the cell, or any combination thereof.

In some embodiments, the one or two chromosomes are “minimized”, whereby non-essential elements have been removed. In some embodiments, the bacteria grow at temperature from about 25° C. to about 42° C. In some embodiments, the growth doubling time is about 5 minutes to 15 minutes. In some embodiments, the minimized chromosome or single chromosome comprises essential elements from Chromosome land Chromosome 2 such that the minimized or single chromosome is capable of supporting survival and replication of the bacteria under non-selective conditions.

In some embodiments, the herein disclosed genetically engineered Vibrio sp. further comprises a heterologous nucleic acid sequence operably linked to a heterologous promoter. In some embodiments the heterologous nucleic acid encodes T7 RNA polymerase. In some embodiments, the heterologous promoter is an inducible promoter. The inducible promoter can be induced by temperature, arabinose, IPTG, etc.

In some embodiments, the invention relates to a method for producing competent Vibrio sp. cells including growing genetically modified Vibrio sp. bacterial cells in a growth-conducive medium; rendering the Vibrio sp. bacterial cells competent; and freezing the cells. In some embodiments, the Vibrio sp. are any of those genetically engineered Vibrio sp. described herein. In some embodiments, rendering the cells competent comprises growing the cells in conducive media supplemented with supplemental salts.

In some embodiments, the present invention relates to a method of producing a biomolecule comprising a) providing a Vibrio sp. having an exogenous nucleic acid that comprises a heterologous nucleic acid sequence encoding the biomolecule. The method can, optionally, include a step of contacting the Vibrio sp. with the exogenous nucleic acid and introducing it into the bacteria; the exogenous nucleic acid can be a plasmid, expression vector, artificial chromosome, or other vector that encodes a heterologous nucleic acid sequence; b) growing the bacteria in a growth-conducive medium wherein the heterologous nucleic acid sequence is expressed, thereby producing the biomolecule; and optionally c) isolating the biomolecule. In some embodiments the exogenous nucleic acid can encode a signal sequence that causes the biomolecule to be secreted from the organism when produced. The biomolecule can therefore be expressed with a signal sequence attached. In some embodiments, the bacteria are any of the genetically engineered Vibrio sp. bacteria disclosed herein, for example a Vibrio natriegens. In some embodiments, the exogenous nucleic acid comprises a nucleic acid sequence encoding Vibrio sp. replication machinery. In some embodiments, the exogenous nucleic acid further comprises an inducible promoter operably linked to the heterologous nucleic acid encoding the biomolecule. In some embodiments, the exogenous nucleic acid includes replication machinery compatible with one or more organisms. In some embodiments the replication machinery is compatible with a heterologous host, such as, for example, E. coli or S. cerevisiae. Alternatively, or additionally, the replication machinery is from V. natriegens. In some embodiments, the heterologous nucleic acid is at least 1 kb or at least 10 kb, or at least 25 kb, or at least 50 kb, or at least 75 kb, or at least 100 kb, or at least 125 kb, or at least 150 kb, or at least 175 kb, or at least 200 kb, or at least 250 kb, or at least 300 kb, or at least 350 kb, or at least 400 kb, or at least 500 kb, or at least 600 kb, or at least 700 kb, or at least 800 kb, or at least 900 kb or at least 1 Mb, or 2 Mb, or 3 Mb, or 5 Mb, or 7 Mb, or 10 kb-1 Mb or 25 kb-1 Mb or 50 kb-1 Mb or 100 kb-1 Mb or 50 kb-2 Mb or 50 kb-3 Mb or 50 kb-5 Mb or 50 kb-7 Mb, or 30 kb-1 Mb or 100 kb-1 Mb or 30 kb-2 Mb or 30 kb-3 Mb or 30 kb-5 Mb or 30 kb-7 Mb or 100 kb-2 Mb or 100 kb-3 Mb or 100 kb-5 Mb or 100 kb-7 Mb. In some embodiments, the heterologous nucleic acid also comprises an inducible promoter, an origin of replication, an origin of transfer, a selectable marker, a counter-selectable marker, a reporter gene, a regulatory element, an enzyme gene, or a combination thereof. In some embodiments, the selectable marker is an antibiotic resistance gene, a gene encoding a polypeptide conferring resistance to a toxin, an auxotrophic marker or a combination thereof. In some embodiments, the antibiotic resistance gene confers resistance to at least one antibiotic such as, for example, bleomycin, carbenicillin, chloramphenicol, gentamycin, glyphosate, hygromycin, kanamycin, neomycin, nourseothricin, phleomycin, puromycin, spectinomycin, streptomycin, tetracycline, or the like. In some embodiments, the reporter gene can be a reporter gene such as, for example a fluorescent protein or beta-galactosidase or the like. In some embodiments, the enzyme can be a nucleic-acid functional enzyme such as, for example, a recombinase, an integrase, a nuclease, a recombineering enzyme, a polymerase, or the like. In some embodiments the recombinase can be Cre recombinase. In some embodiments, the integrase can be at least one of PhiC31 or bxb 1. In some embodiments the nuclease can be a Type II CRISPR Cas9 nuclease. In some embodiments, the polymerase can be a Sp6, T3, or T7 RNA polymerase, or the like. In some embodiments, the inducible promoter can be induced by IPTG, arabinose, temperature, or the like. In some embodiments, the Vibrio sp. bacterial cells can be naturally competent. In some embodiments, the Vibrio sp. cells can be competent cells generated by any of the methods disclosed herein. In some embodiments, the heterologous nucleic acid can be introduced into the cell by conjugation, chemical competence, natural competence, biolistics, transduction, or electroporation. In some embodiments, the growth conducive media can be monitored for the presence of the biomolecule. In other embodiments the heterologous nucleic acid sequence can be unexpressed, but the exogenous vector can be cloned in the Vibrio sp. organism. Growth-conducive media support growth of the organism and examples are provided herein.

In some embodiments, the present disclosure provides a kit comprising competent Vibrio sp. bacterial cells, which can be used for any method any disclosed herein.

### EXAMPLES

### Example 1

### Transformation of V. natriegens with Exogenous DNA Constructs Via Conjugation

This method is used to transfer a mobilizable plasmid into V. natriegens.

Donor preparation: Medium containing appropriate antibiotic is inoculated with a donor strain (containing mobilizable plasmid of interest) and incubated overnight at 37° C. with agitation. Acceptable donor strains include, but are not limited to, strain S17-1 kpir (containing the RP4 conjugation machinery integrated into the chromosome) or EPI300 cells harboring the pRL443 conjugative plasmid.

Recipient preparation: Medium is inoculated with V. natriegens recipient strain and incubated overnight at room temperature with agitation.

Conjugative mating: Donor and recipient cultures are retrieved from incubators. Aliquots of the cultures are centrifuged, supernatants are decanted and washed. Donor and recipient cultures are spotted out on prewarmed LB plates, and incubated. Cells are washed from the LB plate and plated out on glucose plates containing appropriate antibiotic and incubated overnight. Individual V. natriegens colonies that grew on the M9 selective plate are then screened for successful conjugation event via standard methods.

### Example 2

### Transformation of V. natriegens with Exogenous DNA Constructs Via Electroporation

Electroporation protocol: A vial of competent cells of V. natriegens is retrieved from storage and allowed to thaw on ice. Plasmid DNA and electrocompetent cells are combined and mixed. The cell/DNA suspension is transferred to a pre-chilled electroporation cuvette. Cells are electroporated and recovered in recovery media. Aliquots of the recovery media are plated out on pre-warmed agar plates containing appropriate antibiotic. The plates are incubated for several hours to overnight at 30-37° C. for colonies to appear. Cells are screened for successful transformation.

### Example 3

### Transformation of V. natriegens with Exogenous DNA Constructs Via Nucleofection

Electroporation protocol: A vial of competent cells of V. natriegens is retrieved from storage and allowed to thaw on ice. Plasmid DNA and electrocompetent cells are combined and mixed. The cell/DNA suspension is transferred to a pre-chilled electroporation cuvette. Cells are electroporated and recovered in recovery media. Aliquots of the recovery media are plated out on pre-warmed agar plates containing appropriate antibiotic. The plates are incubated for several hours to overnight at 30-37° C. for colonies to appear. Cells are screened for successful transformation.

### Example 4

### Chemical Transformation of V. natriegens with Exogenous DNA Constructs

Cells are prepared using the following protocol:
Prepare a small, overnight culture of the bacteria in LB broth. Grow at 37° C. without shaking.
About 2 h before the main procedure, use 1.0 mL of the overnight culture to inoculate 100 mL of fresh LB broth. This culture is grown with rapid shaking at 37° C. until it reaches roughly 5×10⁷ cells/ml. This corresponds to an OD650 for these cultures, but calibration for each strain is recommended.
Take a 5 mL aliquot of each transformation reaction and transfer to sterile plastic centrifuge tubes. Cool on ice for 10 mm.
Pellet the cells by spinning for 5 mm at 5000 g at 4° C.
Pour off the supernatant and resuspend cells in 25 mL of cold 0.1M CaCl2. Leave on ice for at least 20 min.
Centrifuge for 5 mm at 5000 g at 4° C. A more diffuse pellet is an indication of competent cells.
Resuspend the cells in 0.2 mL of cold 0.1M CaCl2.
Transfer the suspensions to sterile, thin-walled glass bottles or tubes. The use of glass can make the subsequent heat shocks more effective.
To each tube add up to 0.1 mg of DNA, made up in a standard DNA storage buffer such as TE to a volume of 100 mL. Leave on ice for 30 min.
Transfer to a 42° C. water bath for 2 min and return briefly to ice.
Transfer the contents of each tube to 2 mL of LB broth in a small flask. Incubate with shaking at 37° C. for 60-90 min.
Plate 0.1 mL aliquots of undiluted, 10-1 and 10-2 dilutions onto LB plates to which the antibiotics to be used for selection have been added.
Incubate overnight at 37° C.

### Example 5

### Transformation of V. natriegens with Exogenous DNA Constructs Via Natural Competence

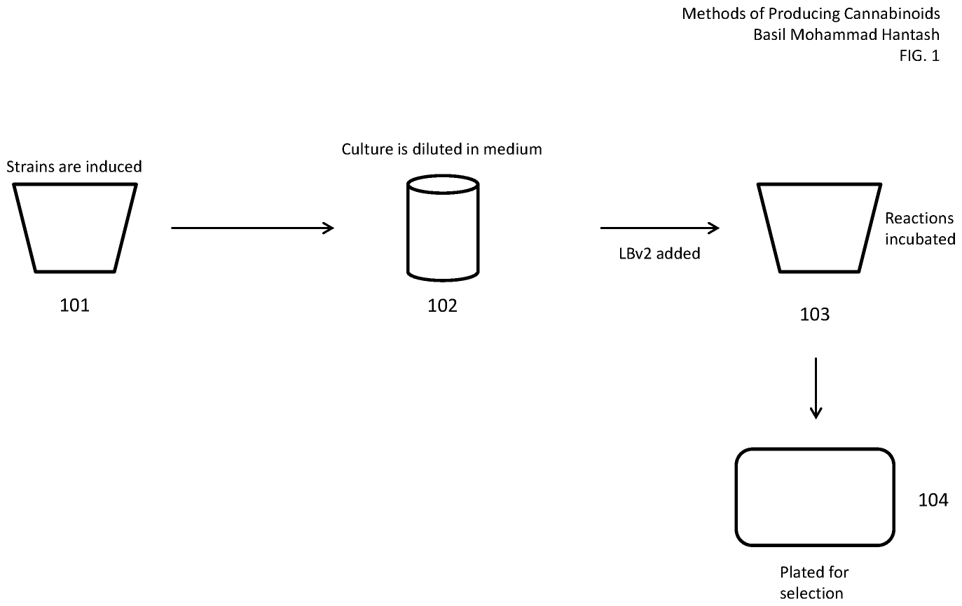

Strains harboring pMMB-tfoX (Vn tfoX or Vc tfoX) are induced to competence by growing overnight (12-18 hours) in LBv2+100 μg/mL carbenicillin+100 μM IPTG in a rollerdrum at 30° C., shown as 101 in FIG. 1. Then, ˜108 CFUs of the overnight culture (˜3.5 μL) are diluted directly into 350 μL of instant ocean medium (28 g/L; Aquarium Systems Inc.) supplemented with 100 μM IPTG. Transforming DNA (tDNA) is then added as indicated, and reactions are incubated statically at 30° C. for 5 hours, 102. Next, 1 mL of LBv2 is added and reactions are outgrown at 30° C. with shaking (250 rpm) for ˜1-2 hrs, 103. Then, reactions are plated for quantitative culture onto media to select for integration of tDNA (i.e. LB+drug=transformants) and onto nonselective media (i.e. plain LB=total viable counts), 104. Transformation efficiency is shown as: transformants/total viable counts.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” When “about” is used in the specification or in a claim, it refers to a number range, varying from the recited number in an amount that, taking into account the number itself and the quality of the characteristic to which the number refers, accounts for variability in measurement or performance that does not change the quality of the numerically-expressed characteristic. In the absence of information to the contrary, “about” can refer to the number plus or minus 0.1%, 0.5%, 1% or 5% of the number itself, as would be appreciated by a person of ordinary skill in the art in reference to the characteristic to which the number refers and the normal quantitative variability around that number that would be understood to not materially change that characteristic. Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### REFERENCES

1. PCT/EP2019/056412
2. Carvalho, Angela et al. “Designing microorganisms for heterologous biosynthesis of cannabinoids.” FEMS yeast research vol. 17, 4 (2017): fox037. doi:10.1093/femsyr/fox037.
3. Pei, Lei, and Markus Schmidt. “Fast-Growing Engineered Microbes: New Concerns for Gain-of-Function Research?.” Frontiers in genetics vol. 9 207. 29 Jun. 2018, doi:10.3389/fgene.2018.00207.
4. Hutchison, Clyde A., et al. “Design and synthesis of a minimal bacterial genome.” Science 351.6280 (2016).
5. Gibson, Daniel G., et al. “Creation of a bacterial cell controlled by a chemically synthesized genome.” Science 329.5987 (2010): 52-56.
6. Lee, Henry H., et al. “Vibrio natriegens, a new genomic powerhouse.” bioRxiv (2016): 058487.
7. Maida, Isabel, et al. “Draft genome sequence of the fast-growing bacterium Vibrio natriegens strain DSMZ 759.” Genome Announc. 1.4 (2013): e00648-13.
8. Weinstock, Matthew T., et al. “Vibrio natriegens as a fast-growing host for molecular biology.” Nature methods 13.10 (2016): 849.
9. Hoffart, Eugenia, et al. “High substrate uptake rates empower Vibrio natriegens as production host for industrial biotechnology.” Appl. environ. microbiol. 83.22 (2017): e01614-17.
10. Pfeifer, Eugen, et al. “Generation of a prophage-free variant of the fast-growing bacterium Vibrio natriegens.” Applied and environmental microbiology 85.17 (2019): e00853-19.
11. Lee, Henry H., et al. “Functional genomics of the rapidly replicating bacterium Vibrio natriegens by CRISPRi.” Nature microbiology 4.7 (2019): 1105-1113.
12. Tschirhart, Tanya, et al. “Synthetic Biology Tools for the Fast-Growing Marine Bacterium Vibrio natriegens.” ACS synthetic biology 8.9 (2019): 2069-2079.
13. Luo, Xiaozhou, et al. “Complete biosynthesis of cannabinoids and their unnatural analogues in yeast.” Nature 567.7746 (2019): 123.
14. Lacoste, Arnaud, Frada Berenshteyn, and Ali H. Brivanlou. “An efficient and reversible transposable system for gene delivery and lineage-specific differentiation in human embryonic stem cells.” Cell stem cell 5.3 (2009): 332-342.
15. Xue, Xingkui, et al. “Stable gene transfer and expression in cord blood-derived CD34+ hematopoietic stem and progenitor cells by a hyperactive Sleeping Beauty transposon system.” Blood, The Journal of the American Society of Hematology 114.7 (2009): 1319-1330.
16. Zhao, Longmei, Takele Teklemariam, and Basil M. Hantash. “Heterelogous expression of mutated HLA-G decreases immunogenicity of human embryonic stem cells and their epidermal derivatives.” Stem cell research 13.2 (2014): 342-354.
17. Schleicher L, Muras V, Claussen B, et al. Vibrio natriegens as Host for Expression of Multisubunit Membrane Protein Complexes. Front Microbiol. 2018; 9:2537. Published 2018 Oct. 25. doi:10.3389/fmicb.2018.02537.
18. Teklemariam, Takele, et al. “Inhibition of DNA methylation enhances HLA-G expression in human mesenchymal stem cells.” Biochemical and biophysical research communications 452.3 (2014): 753-759.
19. Dalia, Triana N et al. “Multiplex Genome Editing by Natural Transformation (MuGENT) for Synthetic Biology in Vibrio natriegens.” ACS synthetic biology vol. 6, 9 (2017): 1650-1655. doi:10.1021/acssynbio.7b00116

1. A genetically modified bacterium for producing a cannabinoid or a cannabinoid derivative.

2. The bacterium of claim 1, wherein the bacterium is E. coli.

3. The bacterium of claim 1, wherein the bacterium is a Vibrio sp.

4. A genetically modified Vibrio sp. bacterium for producing one or more biomolecules.

5. A method of producing a cannabinoid or a cannabinoid derivative, the method comprising:
a) culturing a genetically modified bacterium;
b) recovering the produced cannabinoid or cannabinoid derivative.

6. The method of claim 5, wherein the bacterium is a Vibrio sp.

7. The method of claim 5, wherein the bacterium is E. coli.
